(19) United States
(12) Patent Application Publication
(10) Pub. No.: US 2021/0193263 A1
(43) Pub. Date: Jun. 24, 2021

(54) DESIGNING PROBES FOR DEPLETING ABUNDANT TRANSCRIPTS

(71) Applicant: Illumina, Inc., San Diego, CA (US)

(72) Inventors: Asako Tan, San Diego, CA (US); Robert Kuersten, San Diego, CA (US); Andrew Kennedy, San Diego, CA (US); Jeffrey Koble, San Diego, CA (US)

(21) Appl. No.: 17/125,378

(22) Filed: Dec. 17, 2020

Related U.S. Application Data
(60) Provisional application No. 62/950,891, filed on Dec. 19, 2019.

(51) Int. Cl. G16B 30/10; G16B 40/00; C12Q 1/6876

(52) U.S. Cl. CPC G16B 30/10; C12Q 2600/166; C12Q 1/6876; G16B 40/00

(57) ABSTRACT

Disclosed herein include systems and methods for designing probes for depleting abundant transcripts from a sample. Abundant sequence reads can be determined in a species-agnostic manner, and probes for depleting abundant transcripts can be designed based on the sequences of the top abundant sequences. Also disclosed herein include compositions and kits for depleting abundant transcripts and methods for depleting abundant transcripts.

### CROSS-REFERENCE TO RELATED APPLICATIONS

This application claims the benefit of priority to U.S. Provisional Patent Application No. 62/950,891, filed on Dec. 19, 2019, the content of which is incorporated herein by reference in its entirety.

### BACKGROUND

### Field

This disclosure relates generally to the field of depleting abundant species, and more particularly to designing probes for depleting abundant species.

### Background

One challenge in RNA sequencing for gene expression analysis is that following RNA extraction most of the extracted material is dominated by a small number of highly abundant transcripts, such as the non-coding ribosomal ribonucleic acids (rRNAs). In a total RNA sample from human blood, globin messenger RNAs (mRNAs) can be present at a dominating level. There is a need to deplete abundant transcripts, such as rRNAs and mRNAs, in a sample prior to RNA sequencing.

### SUMMARY

Disclosed herein include embodiments of a system or a method for designing probes for depleting abundant sequences of ribonucleic acid transcripts. In some embodiments, the method is under control of a hardware processor (or a processor, such as a virtual processor) and comprises: receiving a plurality of sequence reads of ribonucleic acid (RNA) transcripts, or products thereof, in a sample. The method can comprise: aligning each of the plurality of sequence reads to a reference nucleotide sequence, or a subsequence thereof, of a plurality of reference nucleotide sequences. The method can comprise: determining abundant sequences of reference nucleotide sequences, or subsequences thereof, of the plurality of reference nucleotide sequences. Each of the abundant sequences can have a coverage above a coverage threshold. The coverage can be related to a number of the sequence reads aligned to the abundant sequence. The method can comprise: determining top abundant sequences, of the abundant sequences of the reference nucleotide sequences with coverages above the coverage threshold, with highest numbers of coverages. The method can comprise: designing one or more nucleic acid probes for depleting each of the top abundant sequences of the reference nucleotide sequences with the highest numbers of coverages based on a sequence of the top abundant sequence, a probe length, and a tiling gap.

In some embodiments, a reference nucleotide sequence of the plurality of reference nucleotide sequences is a reference RNA sequence of a gene. In some embodiments, a reference nucleotide sequence of the plurality of reference nucleotide sequences is a reference deoxyribonucleic acid (DNA) sequence of a gene.

In some embodiments, the coverage threshold is from about 10 to about 10000. In some embodiments, the coverage of an abundant sequence of the abundant sequences is the number of the sequence reads aligned to the abundant sequence. In some embodiments, the coverage of the abundant of the abundant sequences is the minimum number of the sequence reads aligned to each of a plurality of subsequences of the abundant sequence.

In some embodiments, one, at least one, or each abundant sequence of the abundant sequences comprises a plurality of consecutive subsequences of a reference nucleotide sequence of the plurality of reference nucleotide sequences. The number of the sequence reads aligned to each of the plurality of consecutive subsequences can be above the coverage threshold.

In some embodiments, determining the abundant sequences of the reference nucleotide sequences comprises: determining the number of the sequence reads aligned to subsequences of a plurality of subsequences of a reference nucleotide sequence of the plurality of reference nucleotide sequences. Determining the abundant sequences of the reference nucleotide sequences can comprise: determining an abundant sequence of the abundant sequences comprises a plurality of consecutive subsequences of the subsequences of the reference nucleotide sequence. The number of the sequence reads aligned to each of the plurality of consecutive subsequence can be above the coverage threshold.

In some embodiments, one, at least one, or each abundant sequence of the abundant sequences comprises (i) a plurality of subsequences of a reference nucleotide sequence of the plurality of reference nucleotide sequences (ii) and an interspersing subsequence of the reference nucleotide sequence between any two adjacent subsequences of the plurality of subsequences that are not consecutive and are within a threshold distance of each other. The number of the sequence reads aligned to each of the plurality of subsequences can be above the coverage threshold. In some embodiments, the threshold distance is from about 1 nucleotide to about 50 nucleotides in length.

In some embodiments, one, at least one, or each of the plurality of consecutive subsequences, or of the plurality of subsequences, is one nucleotide in length. In some embodiments, one, at least one, or each of the plurality of consecutive subsequences, or of the plurality of subsequences, is at least 10 nucleotides in length.

In some embodiments, determining the abundant sequences of the reference nucleotide sequences comprises: determining putative abundant sequences of the reference nucleotide sequences of the plurality of reference nucleotide sequences each with the coverage above the coverage threshold. Determining the abundant sequences of the reference nucleotide sequences can comprise: determining any two adjacent putative abundant sequences of a reference nucleotide sequence of the reference nucleotide sequences are within a threshold distance on the reference nucleotide sequence. Determining the abundant sequences of the reference nucleotide sequences can comprise: merging the two putative abundant sequences to generate a merged putative abundant sequence comprising the two putative abundant sequences and an interspersing subsequence of the reference nucleotide sequence between the two putative abundant sequences. The abundant sequences can comprise the merged putative abundant sequence and the putative abundant sequences other than the two putative abundant sequences merged. In some embodiments, the method comprises: determining any two adjacent abundant sequences of a reference nucleotide sequence of the reference nucleotide sequences are within a threshold distance on the reference nucleotide sequence; and merging the two abundant sequences to generate a merged abundant sequence comprising the two abundant sequences and an interspersing subsequence of the reference nucleotide sequence between the two abundant sequences. The abundant sequences after the merging can comprise the merged abundant sequence and the abundant sequences before the merging other than the two abundant sequences merged. In some embodiments, the threshold distance is from about 1 nucleotide to about 50 nucleotides in length.

In some embodiments, the highest numbers of coverages comprise from about 10 to about 500 highest numbers of coverages. In some embodiments, the highest numbers of coverages are from about 1% to about 10% of the sequences of reference nucleotide sequences with the coverages above the coverage threshold. In some embodiments, an average length, or a median length, of the sequences with the coverages above the coverage threshold is from about 50 to about 1000 nucleotides in length. In some embodiments, at least 50% to 90% of the sequences with the coverages above the coverage threshold is each at most 200 to 1000 nucleotides in length.

In some embodiments, determining the top abundant sequences of the plurality of reference nucleotide sequences with the coverages above the coverage threshold comprises: sorting the abundant sequences of the plurality of reference nucleotide sequences with the coverages above the coverage threshold into a descending order of the coverages of the abundant sequences. Determining the top abundant sequences of the plurality of reference nucleotide sequences with the coverages above the coverage threshold can comprise: selecting the first abundant sequences in the descending order of the coverages of the abundant sequences as the top abundant sequences. A number of the first abundant sequences in the descending order of the coverages of the abundant sequences can be from about 10 to about 500.

In some embodiments, no two top abundant sequences of the abundant sequences of the reference nucleotide sequences are within a similarity threshold of each other. In some embodiments, the method comprises: determining a similarity score between each pair of the top abundant sequences. The method can comprise: iteratively removing each top abundant sequence having the similarity score, with respect to any other top abundant sequence of the plurality of top abundant sequences remaining, that is above a similarity threshold from the top abundant sequences remaining. In some embodiments, the method comprises: iteratively, determining a similarity score between a pair of the top abundant sequences remaining to be above a similarity threshold; and removing one of the pairs of top abundant sequences from the top abundant sequences remaining. In some embodiments, the similarity threshold is from about 70% to about 90%.

In some embodiments, one, at least one, or each of the one or more nucleic acids comprises RNA, deoxyribonucleic acid (DNA), xeno nucleic acid (XNA), or a combination thereof. The XNA can comprise 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA), Fluoro Arabino nucleic acid (FANA), or a combination thereof.

In some embodiments, the one or more nucleic acid probes for depleting each of the top abundant sequences of the reference nucleotide sequences with the highest numbers of coverages comprise one or more nucleic acid probes tiling the top abundant sequence. Two adjacent probes of the one or more nucleic acid probes can be separated from each other in the top abundant sequence by the tiling gap. In some embodiments, a sequence of one, at least one, or each, of the one or more nucleic acid probes, for depleting each of the top abundant sequences of the reference nucleotide sequences with the highest numbers of coverages, and the top abundant sequence, a subsequence thereof, or reverse complementary sequence of any of the preceding, have a sequence similarity of at least 80%. In some embodiments, the probe length can from about 25 to about 100 nucleotides in length. In some embodiments, the tiling gap is from about 1 to about 50 nucleotides in length. In some embodiments, an average number, or a median number, of the one or more nucleic acid probes for depleting each of the top abundant sequences is from about 1 to about 100. In some embodiments, a total number of the probes designed for depleting the top abundant sequences is fewer than 10000.

In some embodiments, the sample comprises a microbe sample, a microbiome sample, a bacteria sample, a yeast sample, a plant sample, an animal sample, a patient sample, an epidemiology sample, an environmental sample, a soil sample, a water sample, a metatranscriptomics sample, or a combination thereof. In some embodiments, the sample comprises an organism of a species that is not predetermined, an unknown species, or a combination thereof. In some embodiments, the sample comprises organisms of at least two species. The one or more abundant RNA transcripts can comprise RNA transcripts from organisms of at least two species. The sample can comprise at least 10 ng of RNA transcripts.

In some embodiments, one or more abundant RNA transcripts, sequences thereof, or subsequences thereof, have been depleted from the sample using a plurality of depletion probes prior to the RNA transcripts are reverse transcribed to generate complementary DNAs (cDNAs) and the cDNAs, or products thereof, are sequenced to generate the plurality of sequence reads. The one or more abundant RNA transcripts can be ribosomal RNA transcripts and/or globin mRNA transcripts. In some embodiments, no abundant RNA transcript, or any sequence thereof, has been depleted from the sample.

Disclosed herein include embodiments of a system or a method for designing probes for depleting abundant sequences of ribonucleic acid transcripts. In some embodiments, the system comprises: non-transitory memory configured to store executable instructions; and a hardware processor in communication with the non-transitory memory, the hardware processor programmed by the executable instructions to: receive a plurality of sequence reads of ribonucleic acid (RNA) transcripts, or products thereof, in a sample. The hardware processor can be programmed by the executable instructions to: receive a coverage threshold, a probe length, a tiling gap, and/or a maximum number of abundant sequences for depletion. The hardware processor can be programmed by the executable instructions to: align each of the plurality of sequence reads to a reference nucleotide sequence, or a subsequence thereof, of a plurality of reference nucleotide sequences. The hardware processor can be programmed by the executable instructions to: determine abundant sequences of reference nucleotide sequences, or subsequences thereof, of the plurality of reference nucleotide sequences. Each of the abundant sequences can have a coverage above the coverage threshold. The coverage can be related to a number of the sequence reads aligned to the abundant sequence. The hardware processor can be programmed by the executable instructions to: select top abundant sequences, of the abundant sequences of the reference nucleotide sequences with coverages above the coverage threshold, with highest numbers of coverages. A number of the top abundant sequences selected can be at most the maximum number of sequences for depletion. The hardware processor can be programmed by the executable instructions to: design one or more nucleic acid probes for depleting each of the top abundant sequences of the reference nucleotide sequences with the highest numbers of coverages based on a sequence of the abundant sequence, the probe length, and the tiling gap. The hardware processor can be programmed by the executable instructions to: output sequences of the nucleic acid probes for depleting the top abundant sequences designed.

In some embodiments, one or more of the coverage threshold, the probe length, the tiling gap, and/or the maximum number of the abundant sequences for depletion are default values. In some embodiments, one or more of the coverage threshold, the probe length, the tiling gap, and/or the maximum number of the abundant sequences for depletion are non-default values.

In some embodiments, the hardware processor is programmed by the executable instructions to: generate and/or cause to display a first user interface (UI) comprising (i) an input element for receiving a link to the plurality of sequence reads of RNA transcripts, and/or (ii) input elements for receiving the coverage threshold, the probe length, the tiling gap, and/or the maximum number of the abundant sequences for depletion. The first UI can comprise one or more of the default values of the coverage threshold, the probe length, the tiling gap, and/or the maximum number of the abundant sequences for depletion. (i) The plurality of sequence reads of RNA transcripts and/or (ii) the coverage threshold, the probe length, the tiling gap, and/or the maximum number of the abundant sequences for depletion can be received from a user of the system via the first UI.

In some embodiments, to output the sequences of the nucleic acid probes for depleting the top abundant sequences designed, the hardware processor is programmed by the executable instructions to: generate and/or cause to display a second UI comprising (a) sequences of the nucleic acid probes designed, (b) a link to the sequences of the nucleic acid probes designed, and/or (c) an input element for receiving a user input or selection for exporting the sequences of the nucleic acid probes designed.

In some embodiments, a reference nucleotide sequence of the plurality of reference nucleotide sequences is a reference RNA sequence of a gene. In some embodiments, a reference nucleotide sequence of the plurality of reference nucleotide sequences is a reference deoxyribonucleic acid (DNA) sequence of a gene.

In some embodiments, the coverage threshold is from about 10 to about 10000. In some embodiments, the coverage of an abundant sequence of the abundant sequences is the number of the sequence reads aligned to the abundant sequence. In some embodiments, the coverage of the abundant of the abundant sequences is the minimum number of the sequence reads aligned to each of a plurality of subsequences of the abundant sequence.

In some embodiments, one, at least one, or each abundant sequence of the abundant sequences comprises a plurality of consecutive subsequences of a reference nucleotide sequence of the plurality of reference nucleotide sequences. The number of the sequence reads aligned to each of the plurality of consecutive subsequences can be above the coverage threshold.

In some embodiments, to determine the abundant sequences of the reference nucleotide sequences, the hardware processor is programmed by the executable instructions to: determine the number of the sequence reads aligned to subsequences of a plurality of subsequences of a reference nucleotide sequence of the plurality of reference nucleotide sequences; and determine an abundant sequence of the abundant sequences comprises a plurality of consecutive subsequences of the subsequences of the reference nucleotide sequence. The number of the sequence reads aligned to each of the plurality of consecutive subsequence can be above the coverage threshold.

In some embodiments, one, at least one, or each abundant sequence of the abundant sequences comprises (i) a plurality of subsequences of a reference nucleotide sequence of the plurality of reference nucleotide sequences (ii) and an interspersing subsequence of the reference nucleotide sequence between any two adjacent subsequences of the plurality of subsequences that are not consecutive and are within a threshold distance of each other. The number of the sequence reads aligned to each of the plurality of subsequences can be above the coverage threshold. In some embodiments, the threshold distance is from about 1 nucleotide to about 50 nucleotides in length.

In some embodiments, one, at least one, or each of the plurality of consecutive subsequences, or of the plurality of subsequences, is one nucleotide in length. In some embodiments, one, at least one, or each of the plurality of consecutive subsequences, or of the plurality of subsequences, is at least 10 nucleotides in length.

In some embodiments, to determine the abundant sequences of the reference nucleotide sequences, the hardware processor is programmed by the executable instructions to: determine putative abundant sequences of the reference nucleotide sequences of the plurality of reference nucleotide sequences each with the coverage above the coverage threshold; determine any two adjacent putative abundant sequences of a reference nucleotide sequence of the reference nucleotide sequences are within a threshold distance on the reference nucleotide sequence; and merge the two putative abundant sequences to generate a merged putative abundant sequence comprising the two putative abundant sequences and an interspersing subsequence of the reference nucleotide sequence between the two putative abundant sequences. The abundant sequences can comprise the merged putative abundant sequence and the putative abundant sequences other than the two putative abundant sequences merged. In some embodiments, the hardware processor is programmed by the executable instructions to: determine any two adjacent abundant sequences of a reference nucleotide sequence of the reference nucleotide sequences are within a threshold distance on the reference nucleotide sequence; and merge the two abundant sequences to generate a merged abundant sequence comprising the two abundant sequences and an interspersing subsequence of the reference nucleotide sequence between the two abundant sequences. The abundant sequences after the merging can comprise the merged abundant sequence and the abundant sequences before the merging other than the two abundant sequences merged. In some embodiments, the threshold distance is from about 1 nucleotide to about 50 nucleotides in length.

In some embodiments, the highest numbers of coverages comprise from about 10 to about 500 highest numbers of coverages. In some embodiments, the highest numbers of coverages are from about 1% to about 10% of the sequences of reference nucleotide sequences with the coverages above the coverage threshold. In some embodiments, an average length, or a median length, of the sequences with the coverages above the coverage threshold is from about 50 to about 1000 nucleotides in length. In some embodiments, at least 50% to 90% of the sequences with the coverages above the coverage threshold is each at most 200 to 1000 nucleotides in length.

In some embodiments, to determine the top abundant sequences of the plurality of reference nucleotide sequences with the coverages above the coverage threshold, the hardware processor is programmed by the executable instructions to: sort the abundant sequences of the plurality of reference nucleotide sequences with the coverages above the coverage threshold into a descending order of the coverages of the abundant sequences; and select the first abundant sequences in the descending order of the coverages of the abundant sequences as the top abundant sequences. A number of the first abundant sequences in the descending order of the coverages of the abundant sequences can be from about 10 to about 500.

In some embodiments, no two top abundant sequences of the abundant sequences of the reference nucleotide sequences are within a similarity threshold of each other. In some embodiments, the hardware processor is programmed by the executable instructions to: determine a similarity score between each pair of the top abundant sequences; and iteratively remove each top abundant sequence having the similarity score, with respect to any other top abundant sequence of the plurality of top abundant sequences remaining, that is above a similarity threshold from the top abundant sequences remaining. In some embodiments, the hardware processor is programmed by the executable instructions to: iteratively, determine a similarity score between a pair of the top abundant sequences remaining to be above a similarity threshold; and remove one of the pairs of top abundant sequences from the top abundant sequences remaining. In some embodiments, the similarity threshold is from about 70% to about 90%.

In some embodiments, one, at least one, or each of the one or more nucleic acid comprises RNA, deoxyribonucleic acid (DNA), xeno nucleic acid (XNA), or a combination thereof, optionally wherein the XNA comprises 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA), Fluoro Arabino nucleic acid (FANA), or a combination thereof.

In some embodiments, the one or more nucleic acid probes for depleting each of the top abundant sequences of the reference nucleotide sequences with the highest numbers of coverages comprise one or more nucleic acid probes tiling the top abundant sequence. Two adjacent probes of the one or more nucleic acid probes are separated from each other in the top abundant sequence by the tiling gap. In some embodiments, a sequence of one, at least one, or each, of the one or more nucleic acid probes, for depleting each of the top abundant sequences of the reference nucleotide sequences with the highest numbers of coverages, and the top abundant sequence, a subsequence thereof, or reverse complementary sequence of any of the preceding, have a sequence similarity of at least 80%. In some embodiments, the probe length is from about 25 to about 100 nucleotides in length. In some embodiments, the tiling gap is from about 1 to about 50 nucleotides in length. In some embodiments, an average number, or a median number, of the one or more nucleic acid probes for depleting each of the top abundant sequences is from about 1 to about 100. In some embodiments, a total number of the probes designed for depleting the top abundant sequences is fewer than 10000.

In some embodiments, the sample comprises a microbe sample, a microbiome sample, a bacteria sample, a yeast sample, a plant sample, an animal sample, a patient sample, an epidemiology sample, an environmental sample, a soil sample, a water sample, a metatranscriptomics sample, or a combination thereof. In some embodiments, the sample comprises an organism of a species that is not predetermined, an unknown species, or a combination thereof. In some embodiments, the sample comprises organisms of at least two species. The one or more abundant RNA transcripts can comprise RNA transcripts from organisms of at least two species. The sample can comprise at least 10 ng of RNA transcripts.

In some embodiments, one or more abundant RNA transcripts, sequences thereof, or subsequences thereof, have been depleted from the sample using a plurality of depletion probes prior to the RNA transcripts are reverse transcribed to generate complementary DNAs (cDNAs) and the cDNAs, or products thereof, are sequenced to generate the plurality of sequence reads. The one or more abundant RNA transcripts can be ribosomal RNA transcripts and/or globin mRNA transcripts. In some embodiments, no abundant RNA transcript, or any sequence thereof, has been depleted from the sample.

Disclosed herein includes embodiments of a computer readable medium comprising executable instructions that when executed by a hardware processor of a computing system or a device, cause the hardware processor and/or the computing system or the device to perform any method disclosed herein. Disclosed herein includes embodiments of a computer readable medium comprising executable instructions the non-transitory memory is configured to store and/or executed by the hardware processor of any system disclosed herein.

Disclosed herein includes embodiments of a composition for depleting abundant transcripts. In some embodiments, the composition comprises: a plurality of depletion probes; and/or a plurality of supplemental depletion probes comprising nucleic acid probes designed using any method or system disclosed herein. Disclosed herein includes embodiments of a composition for depleting abundant transcripts. In some embodiments, the composition comprises: a plurality of depletion probes comprising nucleic acid probes designed using any method or system disclosed herein. Disclosed herein includes a kit for depleting abundant transcripts. In some embodiments, the kit comprises a composition disclosed herein; and instructions for using the composition to deplete abundant transcripts.

Disclosed herein includes embodiments of a method for depleting abundant transcripts. In some embodiments, the method comprises: receiving a sample comprising a plurality of ribonucleic acid (RNA) transcripts. The method can comprise: depleting abundant transcripts in the sample using a composition disclosed herein and one or more nucleases, to generate a plurality of remaining RNA transcripts in the sample. The method can comprise: performing RNA sequencing of the plurality of remaining RNA transcripts in the sample to generate a plurality of sequencing reads. In some embodiments, the one or more nucleases comprise RNase and/or DNase, optionally wherein the RNase is RNase H, and optionally wherein the DNase is DNase 1.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIGS. 1A-1B are non-limiting exemplary schematic illustrations showing how abundant regions of RNA transcripts in a sample can be determined.

FIG. 2 is a flow diagram showing an exemplary method of designing probes for depleting abundant sequences of ribonucleic acid transcripts.

FIG. 3 is a block diagram of an illustrative computing system configured to design probes for depleting abundant sequences of ribonucleic acid transcripts.

FIGS. 4A-4B are non-limiting exemplary plots showing variable performances of a set of 377 oligonucleotide probes on depleting rRNAs and globin mRNAs across different samples.

FIG. 5 is a non-limiting exemplary plot showing a size distribution of abundant regions in a sample after a set of 377 oligonucleotide probes were used to deplete rRNAs and globin mRNAs.

FIG. 6 is a non-limiting exemplary heatmap showing similarities of abundant regions in a sample after a set of 377 oligonucleotide probes were used to deplete rRNAs and globin mRNAs.

FIG. 7 is a non-limiting exemplary schematic illustration showing in-silico performance of a set of 377 oligonucleotide probes and additional probes designed on depleting rRNAs and globin mRNAs in different samples.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

Disclosed herein include embodiments of a method for designing probes for depleting abundant sequences of ribonucleic acid transcripts. In some embodiments, the method is under control of a hardware processor (or a processor, such as a virtual processor) and comprises: receiving a plurality of sequence reads of ribonucleic acid (RNA) transcripts, or products thereof, in a sample. The method can comprise: aligning each of the plurality of sequence reads to a reference nucleotide sequence, or a subsequence thereof, of a plurality of reference nucleotide sequences. The method can comprise: determining abundant sequences of reference nucleotide sequences, or subsequences thereof, of the plurality of reference nucleotide sequences. Each of the abundant sequences can have a coverage above a coverage threshold. The coverage can be related to a number of the sequence reads aligned to the abundant sequence. The method can comprise: determining top abundant sequences, of the abundant sequences of the reference nucleotide sequences with coverages above the coverage threshold, with highest numbers of coverages. The method can comprise: designing one or more nucleic acid probes for depleting each of the top abundant sequences of the reference nucleotide sequences with the highest numbers of coverages based on a sequence of the top abundant sequence, a probe length, and a tiling gap.

Disclosed herein include embodiments of a system for designing probes for depleting abundant sequences of ribonucleic acid transcripts. In some embodiments, the system comprises: non-transitory memory configured to store executable instructions; and a hardware processor in communication with the non-transitory memory, the hardware processor programmed by the executable instructions to: receive a plurality of sequence reads of ribonucleic acid (RNA) transcripts, or products thereof, in a sample. The hardware processor can be programmed by the executable instructions to: receive a coverage threshold, a probe length, a tiling gap, and/or a maximum number of abundant sequences for depletion. The hardware processor can be programmed by the executable instructions to: align each of the plurality of sequence reads to a reference nucleotide sequence, or a subsequence thereof, of a plurality of reference nucleotide sequences. The hardware processor can be programmed by the executable instructions to: determine abundant sequences of reference nucleotide sequences, or subsequences thereof, of the plurality of reference nucleotide sequences. Each of the abundant sequences can have a coverage above the coverage threshold. The coverage can be related to a number of the sequence reads aligned to the abundant sequence. The hardware processor can be programmed by the executable instructions to: select top abundant sequences, of the abundant sequences of the reference nucleotide sequences with coverages above the coverage threshold, with highest numbers of coverages. A number of the top abundant sequences selected can be at most the maximum number of sequences for depletion. The hardware processor can be programmed by the executable instructions to: design one or more nucleic acid probes for depleting each of the top abundant sequences of the reference nucleotide sequences with the highest numbers of coverages based on a sequence of the abundant sequence, the probe length, and the tiling gap. The hardware processor can be programmed by the executable instructions to: output the sequences of the nucleic acid probes for depleting the top abundant sequences designed.

Disclosed herein includes embodiments of a computer readable medium comprising executable instructions that when executed by a hardware processor of a computing system or a device, cause the hardware processor and/or the computing system or the device to perform any method disclosed herein. Disclosed herein includes embodiments of a computer readable medium comprising executable instructions the non-transitory memory is configured to store and/or executed by the hardware processor of any system disclosed herein.

Disclosed herein includes embodiments of a composition for depleting abundant transcripts. In some embodiments, the composition comprises: a plurality of depletion probes; and/or a plurality of supplemental depletion probes comprising nucleic acid probes designed using any method or system disclosed herein. Disclosed herein includes embodiments of a composition for depleting abundant transcripts. In some embodiments, the composition comprises: a plurality of depletion probes comprising nucleic acid probes designed using any method or system disclosed herein. Disclosed herein includes a kit for depleting abundant transcripts. In some embodiments, the kit comprises a composition disclosed herein; and instructions for using the composition to deplete abundant transcripts.

Disclosed herein includes embodiments of a method for depleting abundant transcripts. In some embodiments, the method comprises: receiving a sample comprising a plurality of ribonucleic acid (RNA) transcripts. The method can comprise: depleting abundant transcripts in the sample using a composition disclosed herein and one or more nucleases, to generate a plurality of remaining RNA transcripts in the sample. The method can comprise: performing RNA sequencing of the plurality of remaining RNA transcripts in the sample to generate a plurality of sequencing reads. In some embodiments, the one or more nucleases comprise RNase and/or DNase, optionally wherein the RNase is RNase H, and optionally wherein the DNase is DNase 1.

Throughout the drawings, reference numbers may be re-used to indicate correspondence between referenced elements. The drawings are provided to illustrate example embodiments described herein and are not intended to limit the scope of the disclosure.

Depleting Abundant Sequences from Samples

One challenge in RNA sequencing for gene expression analysis is that following RNA extraction most of the extracted material is dominated by a small number of highly abundant transcripts, such as the non-coding ribosomal ribonucleic acids (rRNAs). In a total RNA sample from human blood, globin messenger RNAs (mRNAs) can be present at a dominating level.

Wasting the cost of sequencing these few transcripts that can dominate the read depth on an instrument is typically not desirable. For example, in human total RNA samples, the rRNAs can make up to ˜80%-85% of the sequencing reads. A kit, such as called RiboZero (Illumina, San Diego, Calif.), can include probes for depleting rRNA from total RNA samples. The kit can be used to deplete rRNAs and globin mRNAs of one species, such as human, yeast, plant, bacteria. Multiple kits for different species can be needed because rRNAs from different species do not have the same sequences. The further away from each other evolutionarily the species, the more diverse are the rRNA sequences. Therefore, the probes used to hybridize and remove the abundant sequence need to be catered toward the species, or at least a closely related species, in order for the kit to perform well. Costs and logistics for manufacturing the various kits can be high.

A kit, such as RiboZero Plus (Illumina, San Diego, Calif.), can includes probes designed to deplete globin mRNAs and rRNAs of multiple species. The kit can both simplify manufacturing and allow more flexibility in probe design. For example, the kit can be designed to deplete human, mouse and rat rRNAs, human globin mRNAs, and rRNAs from two representative bacterial species (E. coli (gram negative) and B. subtilis (gram positive). The kit can work well for depleting globin mRNAs and rRNAs of these species the kit is designed for.

However, bacteria are very diverse, and a kit designed to deplete globin mRNAs and rRNAs of certain species may not be satisfactory for microbial sequencing in metatranscriptomics, which encompasses microbiome research, environmental microbiology, and epidemiology. The spectrum of bacterial species present in a sample from, for example, soil or gut microbiome may not be predetermined. Further, bacteria species present in a sample can involve hundreds or perhaps thousands of different species. Consequently the probes designed against only two representative bacterial species can be insufficient for the needs of the metatranscriptome field. Furthermore, there is an upper limit to the total number of probes that can be used to deplete abundant transcripts in a sample. Disclosed herein include embodiments of a system and a method for designing probes for depleting abundant sequences (e.g., abundant transcripts, such as rRNAs and globin mRNAs) from a sample, such as a complex sample including a metatranscriptomic biosample.

Designing Probes for Depleting Abundant Sequences from Samples

Disclosed herein include a method for efficient probe design to enable depletion of as many types of abundant sequences of a broad spectrum of species present in a sample, regardless of what species are present in a sample. The method can be used to identify and design probes for the regions or sequences that were poorly depleted. The method can be used to collect, analyze, and design probes to abundant sequences in an unbiased manner. The method can enable agnostic probe design for sample types such as metatranscriptomics sample types. The method can be used for creating a custom probe design tool to provide a user a simple approach to remove any unwanted RNA sequences from their samples.

Bioinformatic analysis of residual rRNA can inform on feasibility of patching depletion gaps through additional or supplemental probes. In some embodiments, abundant sequence reads from a sample depleted of some globin mRNAs and rRNAs using a pool or set of probes are processed, and supplemental probes can be designed based on the abundant sequence reads. The method can be used to identify and design probes for the regions or sequences that were poorly depleted using a pool of probes. The method can be used to collect, analyze, and design probes to abundant sequences in an unbiased manner. Fastq (or another format) file from each sample can be prepared using, for example, SortMeRNA (bioinfolifl.fr/RNA/sortmerna/). The sample can be a metatranscriptomics sample (e.g., a soil, water, or microbiome sample) which can contain a broad spectrum of organisms, many of which may not have been identified.

Globin mRNAs and rRNAs in a sample can be depleted by enzymatic depletion using, for example, one or more nucleases, such as RNase H and DNase 1. The probes can be antisense deoxyribonucleic acid (DNA) oligonucleotides. Each probe can be 50 bases in length. The probes can be tiled across targets with 15-base gaps between probes. The pool can include, for example, 377 probes designed to target: 28S, 18S, 16S, 12S, 5.8S and 5S rRNAs of human, mouse, and rat; five human globin mRNAs; 23S and 16S rRNAs of B. subtilis (a gram-negative bacterium); and 23S and 16S rRNAs of E. coli (a gram-positive bacteria). The 377 probes are referred to herein as the RiboZero+ probes (Illumina, San Diego, Calif.). Nuclease-based RNA depletion using the 377 probes is referred to herein as RiboZero+. The RiboZero+ probes and nuclease-based depletion of abundant transcripts using the RiboZero+ probes have been described in PCT Application No. PCT/US2019/067582, entitled “NUCLEASE-BASED RNA DEPLETION” and filed Dec. 19, 2019, the content of which is incorporated by reference in its entirety. Briefly, DNA probes can hybridize to RNA transcripts to form DNA:RNA hybrids. DNA probes not hybridized to RNA transcripts can be removed. RNase H can be used to degrade regions of the RNA transcripts hybridized to DNA probes in the hybrids and RNA regions adjacent to regions of the RNA transcripts hybridized to DNA probes in the hybrids. DNase I can be used to degrade the remaining DNA probes which previously hybridize to the RNA transcripts in the DNA:RNA hybrids.

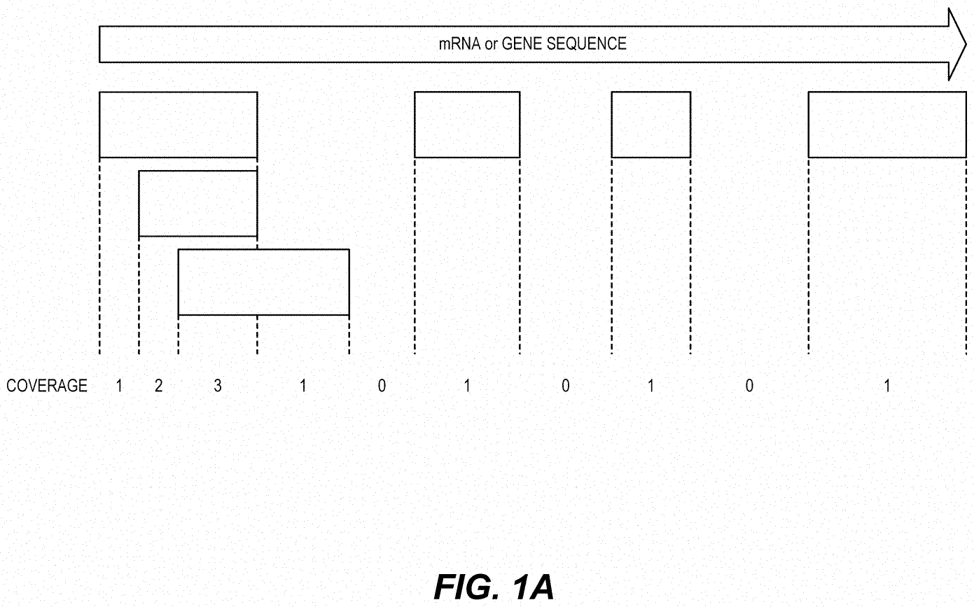

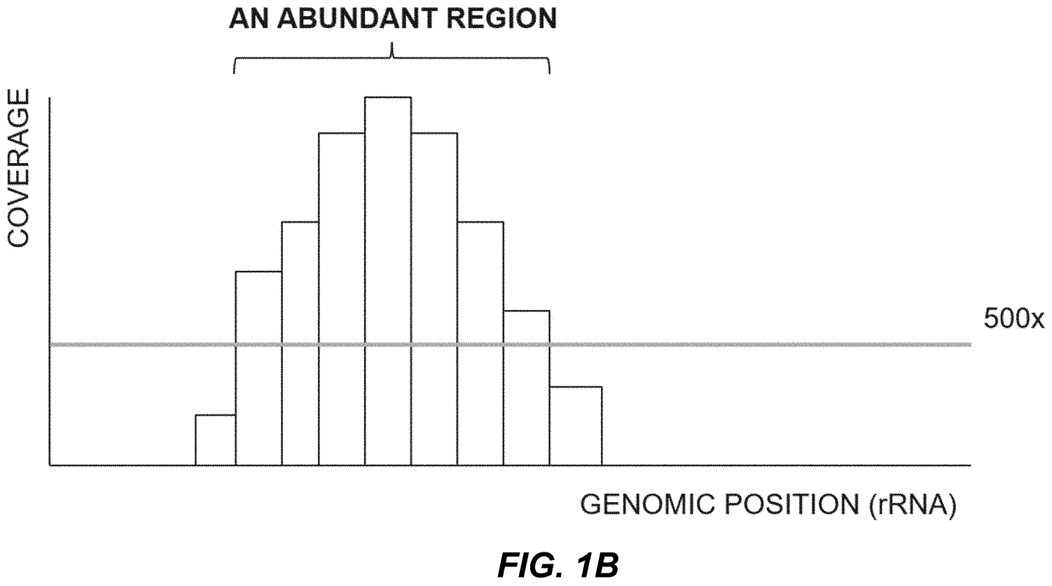

Sequence reads from a sample can be aligned to RNA sequences (e.g., in the publicly available Silva rRNA database) using, for example, SortMeRNA. The file containing the aligned sequences can be processed using, for example, Samtools (samtools.sourceforge.net/). Regions or sequences that are high in coverage, abundance, or read counts (e.g., 500 times or more) can be identified using, for example, Bedtools2 (bedtools.readthedocs.io/en/latest/). FIGS. 1A-1B are non-limiting exemplary schematic illustrations showing how coverages of RNA transcripts in a sample can be determined and abundant regions of RNA transcripts in the sample can be identified. Nearby regions or sequences can be merged (or paired down). After merging, regions or sequences can be sorted or ranked based on the coverages of the regions or sequences. Additional or supplemental probes can be designed based on or targeting top n (e.g., 50) most abundant regions or sequences per sample. Pairwise alignments of the top n (e.g., 50) most abundant regions or sequences can be performed using, for example, Blast (https://blast.ncbi.nlm.nih.gov) to remove regions that are similar to one another. One probe targeting one region likely targets another region with a similar sequence. If two abundant regions have an alignment or similarity score of 80% of more, then one of the two regions can be removed. Supplemental probes can be designed for the remaining regions. Each probe can be 50 bases in length. The probes can be tiled across targets with 15-base gaps between probes. The probes can be DNA oligonucleotides. The probes designed can be synthesized chemically. The probes designed can be added to a pool of probes and/or interchanged with some probes of the pool without major changes to the method of depleting abundant probe sequences.

The probes designed can be used remove abundant transcripts from total RNA samples to allow for greater sensitivity and more cost effective total RNA sequencing applications. The method can be unbiased because the abundant reads, regardless of species the abundant reads come from, can be collected and used to design supplemental probes. There is a limitation on the absolute number of probes that can be pooled and used to obtain sufficient RNA sequencing performance metrics. The method can be used to design probes for efficient depletion while keeping the number of probes to a minimum.

In some embodiments, the method can be quite agnostic. The method may not require the prior identification of a particular species of organism. In some embodiments, the method can collect and process the abundant sequences that escape depletion from existing probes of a probe pool and allow the design of additional probes that can be used to supplement the original probe pool to improve the performance of depletion. In some embodiments, the method allows the design of probes to a broad spectrum of species, yet relies on sequencing reads instead of intact rRNA sequences. In some embodiments, the method can utilize publicly available tools for alignment and data processing, and may not require complex programming. In some embodiments, the method can efficiently design a limited set of probes to keep the cost and complexity of the probe pool to a minimum. In some embodiments, the method can be used to design probes for depleting abundant transcripts in various sample types. The sample types can be highly complex mixtures of different species types, such as eukaryotic and prokaryotic microorganisms such as marine sediment, soil and sludge. Other types of samples include human and mouse gut microbiome.

Example Method of Designing Probes for Depleting Abundant Sequences from Samples

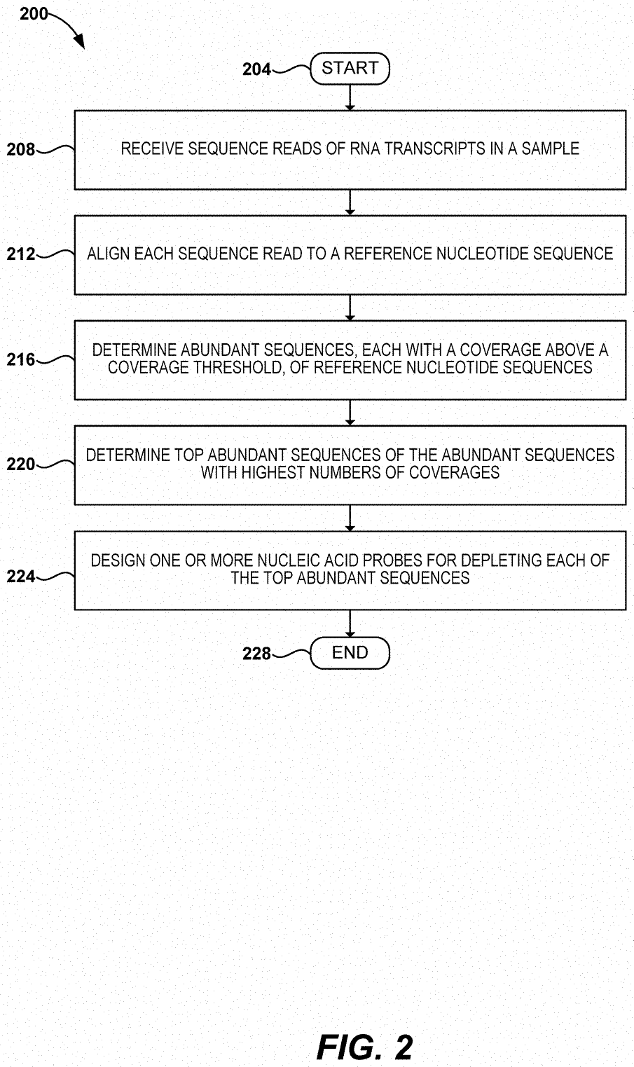

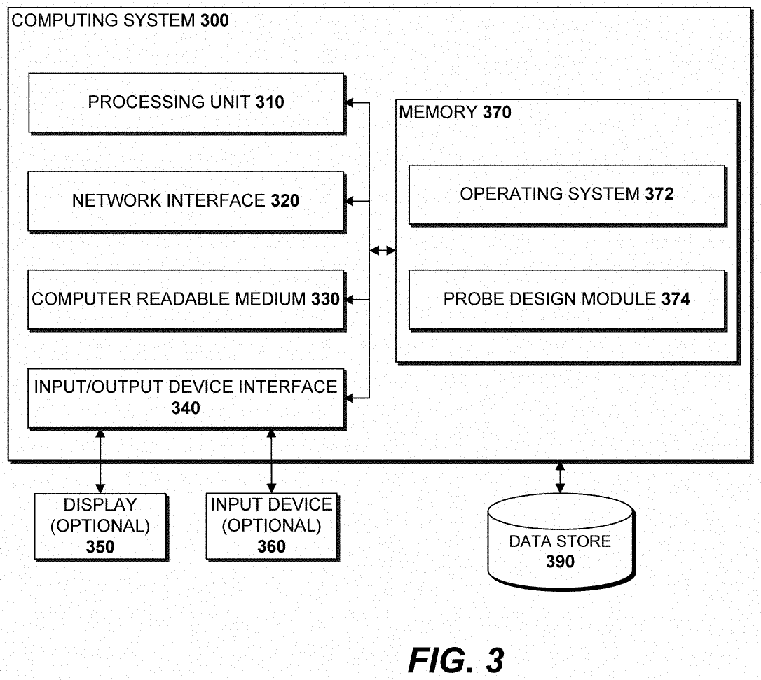

FIG. 2 is a flow diagram showing an exemplary method 200 of designing probes for depleting abundant sequences of nucleic acids such as ribonucleic acid transcripts from samples. The method 200 may be embodied in a set of executable program instructions stored on a computer-readable medium, such as one or more disk drives, of a computing system. For example, the computing system 300 shown in FIG. 3 and described in greater detail below can execute a set of executable program instructions to implement the method 200. When the method 200 is initiated, the executable program instructions can be loaded into memory, such as RAM, and executed by one or more processors of the computing system 300. Although the method 200 is described with respect to the computing system 300 shown in FIG. 3, the description is illustrative only and is not intended to be limiting. In some embodiments, the method 200 or portions thereof may be performed serially or in parallel by multiple computing systems.

After the method 200 begins at block 204, the method 200 proceeds to block 208, where a computing system (e.g., the computing system 300 shown in FIG. 3) receives a plurality of sequence reads of nucleic acids, such as ribonucleic acid (RNA) transcripts, or products thereof (e.g., complementary deoxyribonucleic acid (cDNA) products from first strand synthesis), in a sample.

Sample.

The sample can comprise a microbe sample, a microbiome sample, a bacteria sample, a yeast sample, a plant sample, an animal sample, a patient sample, an epidemiology sample, an environmental sample, a soil sample, a water sample, a metatranscriptomics sample, or a combination thereof. In some embodiments, the sample comprises an organism of a species that is not predetermined, an unknown or unidentified species, or a combination thereof. In some embodiments, the sample comprises organisms of, of about, of at least, or of at most, 1, 2, 3, 4, 5, 6, 7, 8, 9, 10, 20, 30, 40, 50, 60, 70, 80, 90, 100, or a number or a range between any two of these values, species. The one or more abundant RNA transcripts can comprise RNA transcripts from organisms of, of about, of at least, or of at most, 1, 2, 3, 4, 5, 6, 7, 8, 9, 10, 20, 30, 40, 50, 60, 70, 80, 90, 100, or a number or a range between any two of these values, species. The sample can comprise, comprise about, comprise at least, or comprise at most, 1 ng, 2 ng, 3 ng, 4 ng, 5 ng, 6 ng, 7 ng, 8 ng, 9 ng, 10 ng, 20 ng, 30 ng, 40 ng, 50 ng, 60 ng, 70 ng, 80 ng, 90 ng, 100 ng, 200 ng, 300 ng, 400 ng, 500 ng, 600 ng, 700 ng, 800 ng, 900 ng, 1000 ng, of RNA transcripts.

User Inputs.

In some embodiments, the computing system receives a coverage threshold, a probe length, a tiling gap, and/or a maximum number of abundant sequences for depletion from, for example, a user of the system. The computing system can retrieve a coverage threshold, a probe length, a tiling gap, and/or a maximum number of abundant sequences for depletion from, for example, a database of the system, memory of the system, or another system connected with (e.g., directly or indirectly through one or more wired or wireless networks) the system. One or more of the coverage threshold, the probe length, the tiling gap, and/or the maximum number of the abundant sequences for depletion received and/or retrieved can be default or non-default values.

The computing system can generate and/or cause to display a first user interface (UI). The first UI can comprise (i) an input element (e.g., a text box) for receiving a link to the plurality of sequence reads of RNA transcripts, and/or (ii) input elements (e.g., text boxes and/or drop-down lists) for receiving the coverage threshold, the probe length, the tiling gap, and/or the maximum number of the abundant sequences for depletion. The first UI can comprise one or more of the default values of the coverage threshold, the probe length, the tiling gap, and/or the maximum number of the abundant sequences for depletion. (i) The plurality of sequence reads of RNA transcripts and/or (ii) the coverage threshold, the probe length, the tiling gap, and/or the maximum number of the abundant sequences for depletion can be received from a user of the system via the first UI.

Depletion.

One or more abundant RNA transcripts, sequences thereof, or subsequences thereof, can have been depleted from the sample using a plurality of depletion probes prior to the RNA transcripts are reverse transcribed to generate complementary DNAs (cDNAs) and the cDNAs, or products thereof, are sequenced to generate the plurality of sequence reads. For example, some abundant transcripts in the sample, or cells in the sample, may have been depleted using depletion probes. The depletion probes can be designed using the method disclosed herein. The one or more abundant RNA transcripts can be ribosomal RNA transcripts and/or globin mRNA transcripts. In some embodiments, no abundant RNA transcript, or any sequence thereof, has been depleted from the sample.

The method 200 proceeds from block 208 to block 212, where the computing system aligns each of the plurality of sequence reads to a reference nucleotide sequence, or a subsequence thereof, of a plurality of reference nucleotide sequences. A reference nucleotide sequence of the plurality of reference nucleotide sequences can be a reference RNA sequence of a gene, or a subsequence thereof. The reference RNA sequence can be from the Silva rRNA database (www.arb-silva.de). The computing system can align each of the plurality of sequence reads to a reference RNA sequence, or a subsequence thereof, of the plurality of reference RNA sequences using SortMeRNA (bioinfolifl.fr/RNA/sortmerna/). A reference nucleotide sequence of the plurality of reference nucleotide sequences can be a reference deoxyribonucleic acid (DNA) sequence of a gene, or a subsequence thereof.

The method 200 proceeds from block 212 to block 216, where the computing system determines abundant sequences of reference nucleotide sequences, or subsequences thereof, of the plurality of reference nucleotide sequences. Each of the abundant sequences can have a coverage above the coverage threshold. The coverage can be related to a number of the sequence reads aligned to the abundant sequence. The coverage of an abundant sequence of the abundant sequences can be the number of the sequence reads aligned to the abundant sequence. The coverage of the abundant of the abundant sequences can be the minimum number of the sequence reads aligned to each of a plurality of subsequences of the abundant sequence. The number of the sequence reads aligned to each of the plurality of subsequences can be above the coverage threshold. In some embodiments, the coverage threshold is, is about, is at least, or is at most, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or a number or a range between any two of these values.

Subsequences of a Reference Nucleotide Sequence. One, at least one, or each abundant sequence of the abundant sequences can comprise a plurality of consecutive subsequences of a reference nucleotide sequence of the plurality of reference nucleotide sequences. The number of the sequence reads aligned to each of the plurality of consecutive subsequences can be above the coverage threshold.

To determine the abundant sequences of the reference nucleotide sequences, the computing system can determine the number of the sequence reads (e.g., coverage) aligned to subsequences of a plurality of subsequences of a reference nucleotide sequence of the plurality of reference nucleotide sequences. The computing system can determine an abundant sequence of the abundant sequences comprises a plurality of consecutive subsequences of the subsequences of the reference nucleotide sequence. The number of the sequence reads aligned to each of the plurality of consecutive subsequence can be above the coverage threshold.

One, at least one, or each abundant sequence of the abundant sequences can comprise (i) a plurality of subsequences of a reference nucleotide sequence of the plurality of reference nucleotide sequences (ii) and an interspersing subsequence of the reference nucleotide sequence between any two adjacent subsequences of the plurality of subsequences that are not consecutive and are within a threshold distance of each other. For example, if two adjacent abundant sequences have been merged, then the sequence between the two adjacent abundant sequences does not have a high coverage. For example, if three adjacent abundant sequences have been merged, then the resulting abundant subsequence includes two interspersing subsequences between the three adjacent abundant sequences. In some embodiments, the threshold distance is, is about, is at least, or is at most, 1, 2, 3, 4, 5, 6, 7, 8, 9, 10, 20, 30, 40, 50, 60, 70, 80, 90, 100, or a number or a range between any two of these values, nucleotides in length.

One, at least one, or each of the plurality of consecutive subsequences, or of the plurality of subsequences, can be one nucleotide in length. For example, the coverage can be calculated per reference sequence position. One, at least one, or each of the plurality of consecutive subsequences, or of the plurality of subsequences, can be, be about, be at least, or be at most, 1, 2, 3, 4, 5, 6, 7, 8, 9, 10, 20, 30, 40, 50, 60, 70, 80, 90, 100 nucleotides in length. For example, the coverage can be calculated for a stretch of at least 10 nucleotides.

Merging.

Nearby sequences can be merged. To determine the abundant sequences of the reference nucleotide sequences, the computing system can: determine putative abundant sequences of the reference nucleotide sequences of the plurality of reference nucleotide sequences each with the coverage above the coverage threshold. The computing system can determine any two adjacent putative abundant sequences of a reference nucleotide sequence of the reference nucleotide sequences are within a threshold distance on the reference nucleotide sequence. The computing system can merge the two putative abundant sequences to generate a merged putative abundant sequence comprising the two putative abundant sequences and an interspersing subsequence of the reference nucleotide sequence between the two putative abundant sequences. The abundant sequences can comprise the merged putative abundant sequence and the putative abundant sequences other than the two putative abundant sequences merged. In some embodiments, the computing system can determine any two adjacent abundant sequences of a reference nucleotide sequence of the reference nucleotide sequences are within a threshold distance on the reference nucleotide sequence. The computing system can merge the two abundant sequences to generate a merged abundant sequence comprising the two abundant sequences and an interspersing subsequence of the reference nucleotide sequence between the two abundant sequences. The abundant sequences after the merging can comprise the merged abundant sequence and the abundant sequences before the merging other than the two abundant sequences merged. In some embodiments, the threshold distance is, is about, is at least, or is at most, 1, 2, 3, 4, 5, 6, 7, 8, 9, 10, 20, 30, 40, 50, 60, 70, 80, 90, 100, or a number or a range between any two of these values, nucleotides in length.

The method 200 proceeds from block 216 to block 220, where the computing system determines or selects top abundant sequences, of the abundant sequences of the reference nucleotide sequences with coverages above the coverage threshold, with highest numbers of coverages. A number of the top abundant sequences determined or selected can be at most the maximum number of sequences for depletion.

In some embodiments, the highest numbers of coverages comprise, comprise about, comprise at least, or comprise at most, 10, 20, 30, 40, 50, 60, 70, 80, 90, 100, 200, 300, 400, 500, 600, 700, 800, 900, 1000, or a number or a range between any two of these values, highest numbers of coverages. In some embodiments, the highest numbers of coverages are from, from about, from at least, or from at most, 1%, 2%, 3%, 4%, 5%, 6%, 7%, 8%, 9%, 10%, or a number or a range between any two of these values, of the sequences of reference nucleotide sequences with the coverages above the coverage threshold. In some embodiments, an average length, or a median length, of the sequences with the coverages above the coverage threshold is, is about, is at least, or is at most, 50, 60, 70, 80, 90, 100, 200, 300, 400, 500, 600, 700, 800, 900, 1000, or a number or a range between any two of these values, nucleotides in length. In some embodiments, a percentage or a range of percentages (e.g., 50%-90%) of the sequences with the coverages above the coverage threshold each is, is about, is at least, or is at most, 100, 200, 300, 400, 500, 600, 700, 800, 900, 1000, or a number or a range between any two of these values. nucleotides in length. In some embodiments, the percentage or the range of percentages is, is about, is at least, or is at most, 50%, 60%, 70%, 80%, 90%, 100%, or a number or a range between any two of these values.

Sorting. Abundant sequences can be sorted by coverage. In some embodiments, to determine the top abundant sequences of the plurality of reference nucleotide sequences with the coverages above the coverage threshold, the computing system can sort the abundant sequences of the plurality of reference nucleotide sequences with the coverages above the coverage threshold into a descending order of the coverages of the abundant sequences. The computing system can select the first abundant sequences in the descending order of the coverages of the abundant sequences as the top abundant sequences. A number of the first abundant sequences in the descending order of the coverages of the abundant sequences can be, be about, be at least, or be at most, 10, 20, 30, 40, 50, 60, 70, 80, 90, 100, 200, 300, 400, 500, 600, 700, 800, 900, 1000, or a number or a range between any two of these values.

Similar Sequences.

Pairwise alignments of the top abundant sequences can be performed and abundant sequences can be removed such that the remaining abundant sequences are dissimilar. In some embodiments, no two top abundant sequences of the abundant sequences of the reference nucleotide sequences are within a similarity threshold of each other. In some embodiments, the computing system can: determine a similarity score (e.g., a percentage alignment) between each pair of the top abundant sequences; and iteratively remove each top abundant sequence having the similarity score, with respect to any other top abundant sequence of the plurality of top abundant sequences remaining, that is above a similarity threshold from the top abundant sequences remaining. In some embodiments, the computing system can: iteratively, determine a similarity score between a pair of the top abundant sequences remaining to be above a similarity threshold; and remove one of the pairs of top abundant sequences from the top abundant sequences remaining. In some embodiments, the similarity threshold is, is about, is at least, or is at most, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values.

The method 200 proceeds from block 220 to block 224, where the computing system designs one or more nucleic acid probes for depleting each of the top abundant sequences of the reference nucleotide sequences with the highest numbers of coverages based on a sequence of the abundant sequence, the probe length, and the tiling gap.

Probes.

In some embodiments, the one or more nucleic acid probes for depleting each of the top abundant sequences of the reference nucleotide sequences with the highest numbers of coverages comprise one or more nucleic acid probes tiling the top abundant sequence. Two adjacent probes of the one or more nucleic acid probes can be separated from each other in the top abundant sequence by the tiling gap. In some embodiments, a sequence of one, at least one, or each, of the one or more nucleic acid probes, for depleting each of the top abundant sequences of the reference nucleotide sequences with the highest numbers of coverages, and the top abundant sequence, a subsequence thereof, or reverse complementary sequence of any of the preceding, have a sequence similarity of at least 80%. In some embodiments, the sequence similarity is, is about, is at least, or is at most, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values. In some embodiments, the probe length is, is about, is at least, or is at most, 25, 30, 35, 40, 45, 50, 60, 70, 80, 90, 100, or a number or a range between any two of these values, nucleotides in length. In some embodiments, the tiling gap is, is about, is at least, or is at most, 1, 2, 3, 4, 5, 6, 7, 8, 9, 10, 20, 30, 40, 50, or a number or a range between any two of these values, nucleotides in length. In some embodiments, an average number, or a median number, of the one or more nucleic acid probes for depleting each of the top abundant sequences is, is about, is at least, or is at most, 1, 2, 3, 4, 5, 6, 7, 8, 9, 10, 20, 30, 40, 50, 60, 70, 80, 90, 100, or a number or a range between any two of these values. In some embodiments, a total number of the probes designed for depleting the top abundant sequences is, is about, is at least, or is at most, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or a number or a range between any two of these values.

Output.

In some embodiments, the computing system outputs information related to the nucleic acid probes for depleting the top abundant sequences designed. The information related to the nucleic acid probes can include sequences of the nucleic acid probes, the coverage threshold, the probe length, a tiling gap, and/or the maximum number of abundant sequences for depletion. In some embodiments, to output the nucleic acid probes for depleting the top abundant sequences designed, the computing system can generate and/or cause to display a second UI comprising (a) sequences of the nucleic acid probes designed, (b) a link (e.g., a web address) to the sequences of the nucleic acid probes designed, and/or (c) an input element (e.g., a button) for receiving a user input or selection for exporting the sequences of the nucleic acid probes designed.

The method 200 ends at block 228.

### Composition and Kit

Disclosed herein includes embodiments of a composition for depleting abundant transcripts. In some embodiments, the composition comprises: a plurality of depletion probes; and/or a plurality of supplemental depletion probes (e.g., nucleic acid probes, such as DNA probes) designed using any method or system disclosed herein. Disclosed herein includes embodiments of a composition for depleting abundant transcripts. In some embodiments, the composition comprises: a plurality of depletion probes comprising nucleic acid probes designed using any method or system disclosed herein. The depletion probes and/or the supplemental depletion probes can be single stranded nucleic acid probes. Disclosed herein includes a kit for depleting abundant transcripts. In some embodiments, the kit comprises a composition disclosed herein; and instructions for using the composition to deplete abundant transcripts.

In some embodiments, one, at least one, or each of the one or more nucleic acid comprises RNA, deoxyribonucleic acid (DNA), xeno nucleic acid (XNA), or a combination thereof, optionally wherein the XNA comprises 1,5-anhydrohexitol nucleic acid (HNA), cyclohexene nucleic acid (CeNA), threose nucleic acid (TNA), glycol nucleic acid (GNA), locked nucleic acid (LNA), peptide nucleic acid (PNA), Fluoro Arabino nucleic acid (FANA), or a combination thereof.

### Using Probes Designed to Deplete Abundant Transcripts

Disclosed herein includes embodiments of a method for depleting abundant transcripts. In some embodiments, the method comprises: receiving a sample comprising a plurality of ribonucleic acid (RNA) transcripts. The method can comprise: depleting abundant transcripts in the sample using a composition disclosed herein and one or more nucleases, to generate a plurality of remaining RNA transcripts in the sample. The method can comprise: performing RNA sequencing of the plurality of remaining RNA transcripts in the sample to generate a plurality of sequencing reads. In some embodiments, the one or more nucleases comprise RNase and/or DNase. The RNase can be RNase H. The DNase can be DNase 1. In some embodiments, DNA probes of the composition hybridize to RNA transcripts to form DNA:RNA hybrids. Excess DNA probes can be removed. RNase H can be used to degrade regions of the RNA transcripts hybridized to DNA probes in the hybrids and RNA regions adjacent to regions of the RNA transcripts hybridized to DNA probes in the hybrids. DNase I can be used to degrade the remaining DNA probes which previously hybridize to the RNA transcripts in the DNA:RNA hybrids.

### Execution Environment

In FIG. 3 depicts a general architecture of an example computing device 300 configured to implement any probe designing methods disclosed herein. The general architecture of the computing device 300 depicted in FIG. 3 includes an arrangement of computer hardware and software components. The computing device 300 may include many more (or fewer) elements than those shown in FIG. 3. It is not necessary, however, that all of these generally conventional elements be shown in order to provide an enabling disclosure. As illustrated, the computing device 300 includes a processing unit 310, a network interface 320, a computer readable medium drive 330, an input/output device interface 340, a display 350, and an input device 360, all of which may communicate with one another by way of a communication bus. The network interface 320 may provide connectivity to one or more networks or computing systems. The processing unit 310 may thus receive information and instructions from other computing systems or services via a network. The processing unit 310 may also communicate to and from memory 370 and further provide output information for an optional display 350 via the input/output device interface 340. The input/output device interface 340 may also accept input from the optional input device 360, such as a keyboard, mouse, digital pen, microphone, touch screen, gesture recognition system, voice recognition system, gamepad, accelerometer, gyroscope, or other input device.

The memory 370 may contain computer program instructions (grouped as modules or components in some embodiments) that the processing unit 310 executes in order to implement one or more embodiments. The memory 370 generally includes RAM, ROM and/or other persistent, auxiliary or non-transitory computer-readable media. The memory 370 may store an operating system 372 that provides computer program instructions for use by the processing unit 310 in the general administration and operation of the computing device 300. The memory 370 may further include computer program instructions and other information for implementing aspects of the present disclosure.

For example, in one embodiment, the memory 370 includes a probe design module 374 for designing probes, such as the method 200 for designing probes for depleting abundant sequences described with reference to FIG. 2. In addition, memory 370 may include or communicate with the data store 390 and/or one or more other data stores that store the sequencing reads used to design probes and/or the probes designed.

### EXAMPLE

Some aspects of the embodiments discussed above are disclosed in further detail in the following example, which are not in any way intended to limit the scope of the present disclosure.

### Example 1

### Probe Design

This example demonstrates designing probes for depleting abundant sequences from samples.

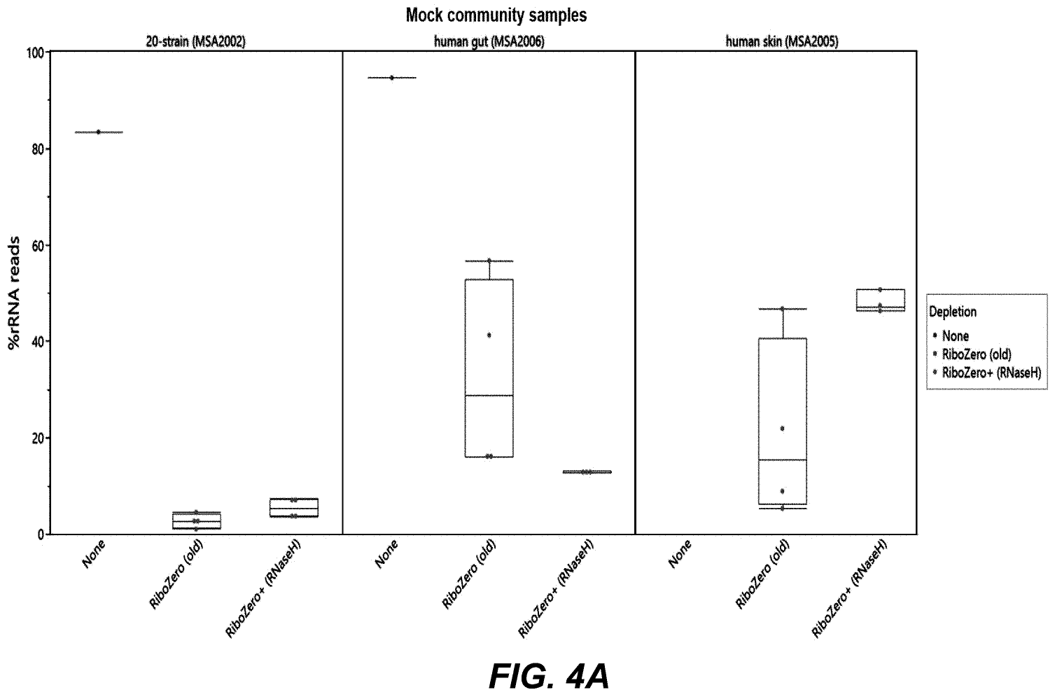

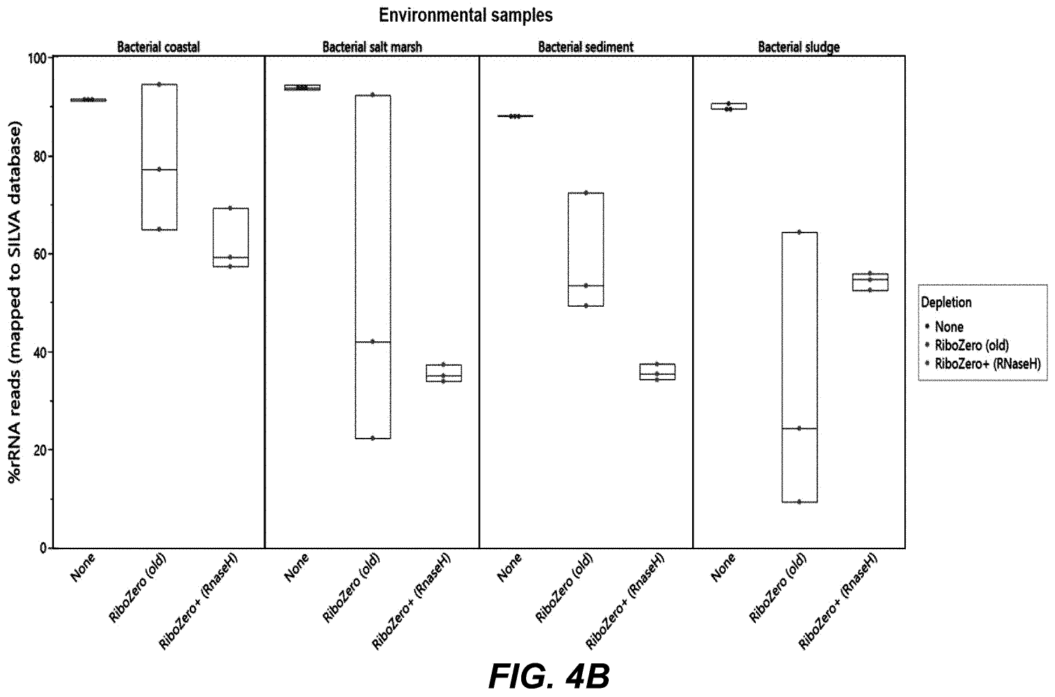

FIGS. 4A-4B are non-limiting exemplary plots showing variable performances of RiboZero and the set of 377 depletion probes of RiboZero+ on depleting rRNAs and globin mRNAs across different samples. The set of 377 depletion probes were used to deplete globin mRNAs and rRNAs in mock community samples from American Type Culture Collection (FIG. 4A) and metatranscriptomics RNA samples from several environments (FIG. 4B), including marine sludge, coastal, sediment, and salt marsh. The samples were sequenced using TruSeq (Illumina, San Diego, Calif.) stranded RNA kits. rRNA depletion was good for some samples and not other samples. Without being limited by theory, the different levels of depletion observed were due to bacterial rRNA regions that the probes do not effectively hybridize to and therefore do not deplete efficiently. FIGS. 4A-4B show that RiboZero+ had higher precisions in all samples tested and variable ribodepletion performance across sample types. RiboZero outperformed in a human skin sample and a 20-strain mock community, and an environmental (bacterial) sludge sample. RiboZero+ (RNaseH) had superior performance on a human gut mock community, and environmental (bacterial) coastal and sediment samples. The RiboZero+ method was uniquely capable of facile performance upgrades or sample extensions.

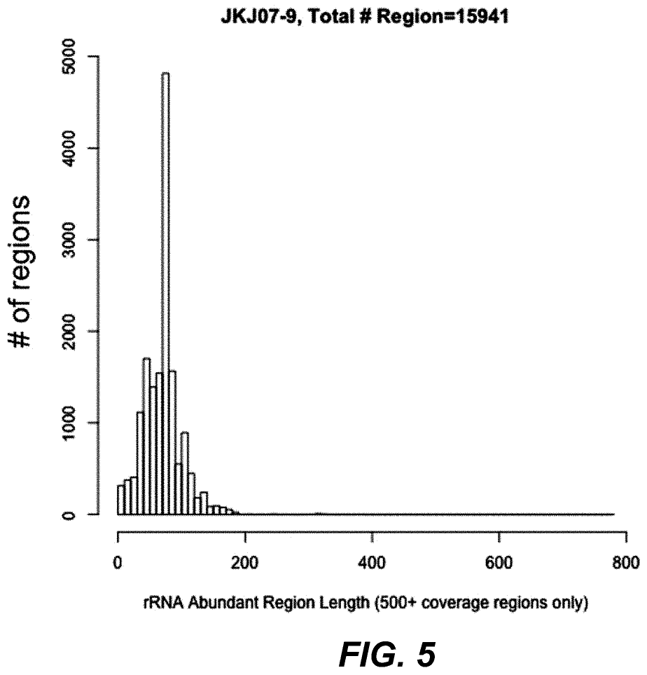

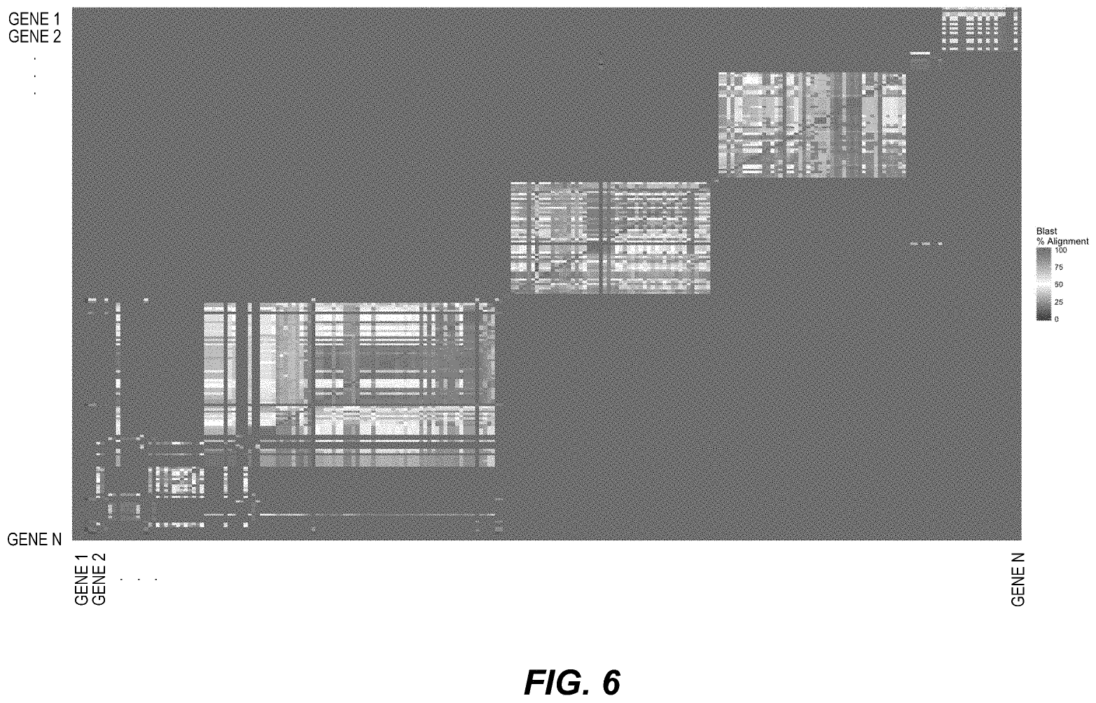

Supplemental probes were designed for mock samples from American Type Culture Collection (20 Strain Mix (MSA2002)—8 replicates; Skin Mix (MSA2005)—6 replicates; and Gut Mix (MSA 2006)—6 replicates) and environmental samples (coastal, sediment, sludge, and salt marsh—2 replicates each). The RiboZero+ probes were used to deplete abundant transcripts in the samples. The remaining rRNA sequences were sequenced using TruSeq (Illumina, San Diego, Calif.) stranded RNA kits. Fastq (or another format) file from each sample was prepared using SortMeRNA (bioinfolifl.fr/RNA/sortmerna/). Sequence reads from a sample were aligned to RNA sequences in the publicly available Silva rRNA database using SortMeRNA. The file containing the aligned sequences was processed using Samtools (samtools.sourceforge.net/). Regions or sequences that were high in coverage, abundance, or read counts (500 times or more) were identified using Bedtools2 (bedtools.readthedocs.io/en/latest/). FIG. 5 is a non-limiting exemplary plot showing a size distribution of abundant regions with coverages of at least 500 in a sample after the RiboZero+ probes were used to deplete rRNAs and globin mRNAs. Most of the regions or sequences that were high in coverage were under 200 nucleotides in length as shown in FIG. 5. Nearby regions or sequences were merged (or paired down). After merging, regions or sequences were sorted or ranked based on the coverages of the regions or sequences. Additional or supplemental probes were designed to target top 50 most abundant regions or sequences per sample. Pairwise alignments of the top 50 most abundant regions or sequences were performed using Blast (https://blast.ncbi.nlm.nih.gov) to remove regions that are similar to one another. If two abundant regions had an alignment percentage of 80% of more, then one of the two regions were removed. FIG. 6 is a non-limiting exemplary heatmap showing similarities of abundant regions in the sample after depletion using RiboZero+. The heatmap shows blocks of similar sequences where minimal and focused sets of probes can be designed. Supplemental probes were designed for the remaining regions. The probes were designed to be 50 nucleotides in length and to tile across targets with 15-base gaps between probes. For the gut sample type, 50 supplemental probes were designed. For the skin sample type, 56 supplemental probes were designed. For the mix sample type of 20 strains, 274 supplemental probes remained after about 50 of the designed probes were paired down. A total of 380 supplemental probes were designed for the gut sample type, the skin sample type, and the mixed sample type of 20 strains. For the environmental sample type, 179 probes were designed.

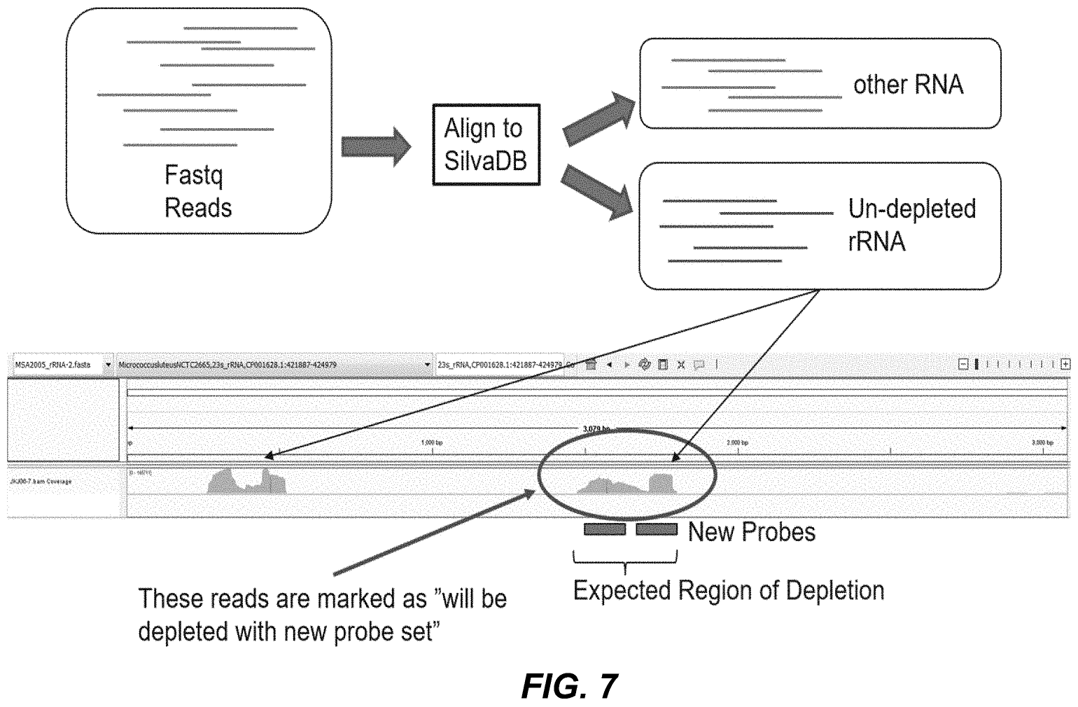

After probe sequences were generated for each sample type, the probe sequences were analyzed in silico to assess how well the probes work. FIG. 7 is a non-limiting exemplary schematic illustration of determining in-silico performance of the RiboZero+ probes and supplemental probes designed on depleting rRNAs and globin mRNAs in different samples. Blast was performed on the supplemental or new probe sequences against the Silva Database. The Blast result was filtered (with % alignment of at least 80) and a 50-base pair padding was added on each end. A padding was added on each end of the Blast hit regions as the probes were expected to work around the region, not just where the probe binds. A “Region New Probes Can Deplete” included the region each probe binds and the two padding on the two ends of the probe. For each sequenced sample, SortMeRNA was run (keep only the best hit) to obtain the rRNA alignment against Silva Database. The reads that overlapped with “Region New Probes Can Deplete” were counted using Bedtools2. The numbers of reads that originally mapped to rRNA and then potentially can be depleted by the new probe set were estimated. Tables 1-4 show the performance of the supplemental probes designed.

TABLE 1
Gut samples (50 supplemental probes)

| Sample | Original rRNA Content | Estimate % rRNA with New Probes |
|---|---|---|
| 1 | 15.46% | 4.13% |
| 2 | 14.58% | 3.22% |
| 3 | 14.9% | 3.7% |
| 4 | 10.87% | 3.06% |
| 5 | 11.04% | 2.96% |
| 6 | 9.15% | 1.38% |

TABLE 2
Skin samples (56 supplemental probes)

| Sample | Original rRNA Content | Estimate % rRNA with New Probes |
|---|---|---|
| 1 | 49.68% | 6.58% |
| 2 | 52.94% | 7.31% |
| 3 | 48.66% | 6.65% |
| 4* | 56.15% | 32.38% |
| 5 | 57.19% | 5% |
| 6 | 55.83% | 3.27% |

*Sample 4 had a very low yield (16k reads total compared to over 1M reads for others)

TABLE 3
Mix samples of 20 strains (274 supplemental probes)

| Sample | Original rRNA Content | Estimate % rRNA with New Probes |
|---|---|---|
| 1 | 18.25% | 5.51% |
| 2 | 19.08% | 5.31% |
| 3 | 8.00% | 1.70% |
| 4 | 10.11% | 4.61% |
| 5 | 7.24% | 3.48% |
| 6 | 5.84% | 1.72% |
| 7 | 4.09% | 1.62% |

TABLE 4
Environmental samples (179 supplemental probes)

| | | Original | Estimate % rRNA |
|---|---|---|---|
| Sample | Environment | rRNA Content | with New Probes |
| 1 | Coastal | 60.23% | 40.74% |
| 2 | Coastal | 61.89% | 44.03% |
| 3 | Sediment | 53.15% | 45.3% |
| 4 | Sediment | 55.30% | 48.16% |
| 5 | Sludge | 63.96% | 51.27% |
| 6 | Sludge | 63.06% | 49.94% |
| 7 | Salt Marsh | 52.02% | 45.81% |
| 8 | Salt Marsh | 42.76% | 35.36% |

Altogether, these data show that the supplemental probes designed using the method disclosed herein can have good performance in depleting abundant transcripts in different samples.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A method for designing probes for depleting abundant sequences of ribonucleic acid transcripts comprising:
under control of a hardware processor:
receiving a plurality of sequence reads of ribonucleic acid (RNA) transcripts, or products thereof, in a sample;
aligning each of the plurality of sequence reads to a reference nucleotide sequence, or a subsequence thereof, of a plurality of reference nucleotide sequences;
determining abundant sequences of reference nucleotide sequences, or
subsequences thereof, of the plurality of reference nucleotide sequences, wherein each of the abundant sequences has a coverage, related to a number of the sequence reads aligned to the abundant sequence, above a coverage threshold;
determining top abundant sequences, of the abundant sequences of the reference nucleotide sequences with coverages above the coverage threshold, with highest numbers of coverages; and
designing one or more nucleic acid probes for depleting each of the top abundant sequences of the reference nucleotide sequences with the highest numbers of coverages based on a sequence of the top abundant sequence, a probe length, and a tiling gap.

2.-4. (canceled)

5. The method of claim 1, wherein the coverage of an abundant sequence of the abundant sequences is the number of the sequence reads aligned to the abundant sequence, or wherein the coverage of the abundant of the abundant sequences is the minimum number of the sequence reads aligned to each of a plurality of subsequences of the abundant sequence.

6. The method of claim 1, wherein one, at least one, or each abundant sequence of the abundant sequences comprises a plurality of consecutive subsequences of a reference nucleotide sequence of the plurality of reference nucleotide sequences, and wherein the number of the sequence reads aligned to each of the plurality of consecutive subsequences is above the coverage threshold.

7. The method of claim 1, wherein determining the abundant sequences of the reference nucleotide sequences comprises:
determining the number of the sequence reads aligned to subsequences of a plurality of subsequences of a reference nucleotide sequence of the plurality of reference nucleotide sequences; and
determining an abundant sequence of the abundant sequences comprises a plurality of consecutive subsequences of the subsequences of the reference nucleotide sequence, wherein the number of the sequence reads aligned to each of the plurality of consecutive subsequence is above the coverage threshold.

8. The method of claim 1, wherein one, at least one, or each abundant sequence of the abundant sequences comprises (i) a plurality of subsequences of a reference nucleotide sequence of the plurality of reference nucleotide sequences (ii) and an interspersing subsequence of the reference nucleotide sequence between any two adjacent subsequences of the plurality of subsequences that are not consecutive and are within a threshold distance of each other, and wherein the number of the sequence reads aligned to each of the plurality of subsequences is above the coverage threshold.

9. (canceled)

10. (canceled)

11. The method of claim 1, wherein determining the abundant sequences of the reference nucleotide sequences comprises:
determining putative abundant sequences of the reference nucleotide sequences of the plurality of reference nucleotide sequences each with the coverage above the coverage threshold;
determining any two adjacent putative abundant sequences of a reference nucleotide sequence of the reference nucleotide sequences are within a threshold distance on the reference nucleotide sequence; and
merging the two putative abundant sequences to generate a merged putative abundant sequence comprising the two putative abundant sequences and an interspersing subsequence of the reference nucleotide sequence between the two putative abundant sequences, wherein the abundant sequences comprise the merged putative abundant sequence and the putative abundant sequences other than the two putative abundant sequences merged.

12. The method of claim 1, comprising:
determining any two adjacent abundant sequences of a reference nucleotide sequence of the reference nucleotide sequences are within a threshold distance on the reference nucleotide sequence; and
merging the two abundant sequences to generate a merged abundant sequence comprising the two abundant sequences and an interspersing subsequence of the reference nucleotide sequence between the two abundant sequences, wherein the abundant sequences after the merging comprise the merged abundant sequence and the abundant sequences before the merging other than the two abundant sequences merged.

13.-17. (canceled)

18. The method of claim 1, wherein determining the top abundant sequences of the plurality of reference nucleotide sequences with the coverages above the coverage threshold comprises:
sorting the abundant sequences of the plurality of reference nucleotide sequences with the coverages above the coverage threshold into a descending order of the coverages of the abundant sequences; and
selecting the first abundant sequences in the descending order of the coverages of the abundant sequences as the top abundant sequences, optionally wherein a number of the first abundant sequences in the descending order of the coverages of the abundant sequences is from about 10 to about 500.

19. (canceled)

20. The method of claim 1, comprising:
determining a similarity score between each pair of the top abundant sequences; and
iteratively removing each top abundant sequence having the similarity score, with respect to any other top abundant sequence of the plurality of top abundant sequences remaining, that is above a similarity threshold from the top abundant sequences remaining.

21. The method of claim 1, comprising: iteratively,
determining a similarity score between a pair of the top abundant sequences remaining to be above a similarity threshold; and
removing one of the pairs of top abundant sequences from the top abundant sequences remaining.

22.-29. (canceled)

30. The method of claim 1, wherein the sample comprises an organism of a species that is not predetermined, an unknown species, or a combination thereof.

31. The method of claim 1, wherein the sample comprises organisms of at least two species, and/or wherein the one or more abundant RNA transcripts comprise RNA transcripts from organisms of at least two species.

32. (canceled)

33. The method of claim 1, wherein one or more abundant RNA transcripts, sequences thereof, or subsequences thereof, have been depleted from the sample using a plurality of depletion probes prior to the RNA transcripts are reverse transcribed to generate complementary DNAs (cDNAs) and the cDNAs, or products thereof, are sequenced to generate the plurality of sequence reads.

34. The method of claim 33, wherein the one or more abundant RNA transcripts are ribosomal RNA transcripts and/or globin mRNA transcripts.

35. The method of claim 1, wherein no abundant RNA transcript, or any sequence thereof, has been depleted from the sample.

36. A system for designing probes for depleting abundant sequences of ribonucleic acid transcripts comprising:
non-transitory memory configured to store executable instructions; and
a hardware processor in communication with the non-transitory memory, the hardware processor programmed by the executable instructions to:
receive a plurality of sequence reads of ribonucleic acid (RNA) transcripts, or products thereof, in a sample;
receive a coverage threshold, a probe length, a tiling gap, and/or a maximum number of abundant sequences for depletion;
align each of the plurality of sequence reads to a reference nucleotide sequence, or a subsequence thereof, of a plurality of reference nucleotide sequences;
determine abundant sequences of reference nucleotide sequences, or subsequences thereof, of the plurality of reference nucleotide sequences, wherein each of the abundant sequences hash a coverage, related to a number of the sequence reads aligned to the abundant sequence, above the coverage threshold;
select top abundant sequences, of the abundant sequences of the reference nucleotide sequences with coverages above the coverage threshold, with highest numbers of coverages, wherein a number of the top abundant sequences selected is at most the maximum number of sequences for depletion;
design one or more nucleic acid probes for depleting each of the top abundant sequences of the reference nucleotide sequences with the highest numbers of coverages based on a sequence of the abundant sequence, the probe length, and the tiling gap; and
output sequences of the nucleic acid probes for depleting the top abundant sequences designed.

37. (canceled)

38. The system of claim 36, wherein the hardware processor is programmed by the executable instructions to: generate and/or cause to display a first user interface (UI) comprising (i) an input element for receiving a link to the plurality of sequence reads of RNA transcripts, and/or (ii) input elements for receiving the coverage threshold, the probe length, the tiling gap, and/or the maximum number of the abundant sequences for depletion, and wherein (i) the plurality of sequence reads of RNA transcripts and/or (ii) the coverage threshold, the probe length, the tiling gap, and/or the maximum number of the abundant sequences for depletion are received from a user of the system via the first UI.

39. The system of claim 36, wherein to output the sequences of the nucleic acid probes for depleting the top abundant sequences designed, the hardware processor is programmed by the executable instructions to: generate and/or cause to display a second UI comprising (a) sequences of the nucleic acid probes designed, (b) a link to the sequences of the nucleic acid probes designed, and/or (c) an input element for receiving a user input or selection for exporting the sequences of the nucleic acid probes designed.

40.-73. (canceled)

74. A composition for depleting abundant transcripts comprising:
nucleic acid probes designed using the method of claim 1.

75. (canceled)

76. (canceled)

77. A method for depleting abundant transcripts comprising:
receiving a sample comprising a plurality of ribonucleic acid (RNA) transcripts;
depleting abundant transcripts in the sample using a composition of claim 74 and one or more nucleases, to generate a plurality of remaining RNA transcripts in the sample; and
performing RNA sequencing of the plurality of remaining RNA transcripts in the sample to generate a plurality of sequencing reads.

78. (canceled)
